(19) United States
(12) Patent Application Publication
(10) Pub. No.: US 2007/0048226 A1
(43) Pub. Date: Mar. 1, 2007

(54) Biochemical marker detection device

(75) Inventors: Gerald E. Loeb, South Pasadena, CA (US); Laura Marcu, Sierra Madre, CA (US); Sanmao Kang, Shanghai (CN); Kuo-Chih (Vincent) Liao, Pasadena, CA (US)

Correspondence Address: McDERMOTT WILL & EMERY, LLP, 34th Floor, 2049 Century Park East, Los Angeles, CA, 90067, US

(73) Assignee: Alfred E.Mann Institute for Biomedical Engineering at the University of Southern California

(21) Appl. No.: 11/350,695

(22) Filed: Feb. 8, 2006

Related U.S. Application Data
(60) Provisional application No. 60/651,319, filed on Feb. 9, 2005.

(51) Int. Cl. A61K 49/00; C12Q 1/00

(52) U.S. Cl. 424/9.6; 435/4

(57) ABSTRACT

A probe device for detecting chemotherapy effectiveness, and methods of use are disclosed. The device includes a fiber optic probe element that can be injected into a tumor. The probe element is connected to an external controlling/measurement element, which injects a reagent through the probe and into the tumor. The reagent reacts with biological markers indicative of chemotherapy effectiveness.

### CROSS REFERENCE TO RELATED APPLICATIONS

This patent application is related to and claims the benefit of the filing date of U.S. provisional application Ser. No. 60/651,319, filed Feb. 9, 2005, entitled “Method and Apparatus to Detect the Expression of Biochemical Markers on Cell Surfaces;” the contents of which are incorporated herein by reference.

### BACKGROUND OF THE INVENTION

1. Field of the Invention

The present invention relates generally to devices and methods for detection of chemotherapy effectiveness.

2. General Background and State of the Art: Advances in genetics and molecular biology are providing many new chemotherapeutic agents that are much more selective in their effects on specific tumors and cell types rather than generally cytotoxic (e.g. Erbitux, Avastin, Tarceva, etc.). The faster and more reliably the response of the tumor cells can be measured, the more options can be explored practically by the physician or researcher. While the selection of the correct cancer chemotherapeutic agent and the identification of its minimal therapeutic dose are critical for safe and effective treatment, this is typically done by observing the clinical response of a tumor (e.g. overall size) to various therapeutic trials.

### SUMMARY

In one aspect of the biochemical detection systems and methods, a device and system to detect the effectiveness of chemotherapy agents comprise a probe that can be inserted into and maintained in a target tissue of the body. When connected to external apparatus as described herein, this probe can be used to detect the appearance of molecules indicative of apoptosis on cells of the tissue in the vicinity of the end of the probe.

In another aspect of the biochemical detection systems and methods, a method to detect the effectiveness of chemotherapy agents comprises inferring changes in the rate of diffusion of a fluorescent reagent through tissue by releasing the reagent locally into the tissue and measuring the fluorescence of the reagent in the immediate vicinity of the point of release via one or more optical fibers. The fluorescent reagent binds to markers on the surface of cancer cells indicative of apoptosis. In some embodiments, the markers comprise cell adhesion molecules. In an exemplary embodiment, the marker includes phosphatidyl serine. Embodiments of the biochemical detection systems and methods can be used in vivo and/or in vitro.

In yet another aspect of the biochemical detection systems and methods, a system for measuring the effectiveness of chemotherapy agents comprises a probe and a control/measurement apparatus, wherein the probe is thin and flexible enough to facilitate placement and fixation in a target tissue of the body and percutaneous passage to the external apparatus for making measurements. The control/measurement apparatus is located outside of the body. The probe comprises at least one hollow port (i.e. microcapillary) that can be filled with a fluorescent reagent to be measured. The control/measurement apparatus is adapted to propel that reagent from the end of the probe at a controllable rate. The probe comprises at least one optical fiber that can be used to convey photons inward to excite the fluorophor and to convey fluorescence outward for measurement by the control/measurement apparatus. Further embodiments comprise the use of a plurality of optical fibers, such as two optical fibers for example, to separate the excitation and fluorescent light.

In yet another aspect of the biochemical detection systems and methods, the fluorescent reagents used to bind to phosphatidyl serine comprise annexin-V and/or FM1-43. In another aspect of the invention, the fluorescent reagents used to bind to CAMs comprise immunofluorescent agents for NCAMs.

In still a further aspect of the invention, the biochemical detection systems and methods allow pulsed release of fluorescent reagent and measurement of temporal features of fluorescence. In exemplary embodiments, electrophoretic voltage or hydrostatic pressure are used to control extrusion of the fluorescent reagent.

It is understood that other embodiments of the biochemical detection systems and methods will become readily apparent to those skilled in the art from the following detailed description, wherein it is shown and described only exemplary embodiments of the of the biochemical detection systems and methods by way of illustration. As will be realized, the of the biochemical detection systems and methods are capable of other and different embodiments and its several details are capable of modification in various other respects, all without departing from the spirit and scope of the biochemical detection systems and methods. Accordingly, the drawings and detailed description are to be regarded as illustrative in nature and not as restrictive.

### BRIEF DESCRIPTION OF THE DRAWINGS

Aspects of the present invention are illustrated by way of example, and not by way of limitation, in the accompanying drawings, wherein:

FIG. 1 is a schematic drawing illustrating an exemplary mode of an external control/measuring apparatus connected to a probe which is implanted into a tumor in a body;

FIG. 2 illustrates a the distal end of an exemplary probe inserted into a tumor;

FIG. 3 represents an exemplary mode of operation of a preferred embodiment of the device and system;

FIG. 4 illustrates an exemplary configuration of the device and system for in vitro use.

### DETAILED DESCRIPTION

The detailed description set forth below is intended as a description of exemplary embodiments of the biochemical detection systems and methods and is not intended to represent the only embodiments in which the biochemical detection systems and methods can be practiced. The term “exemplary” used throughout this description means “serving as an example, instance, or illustration,” and should not necessarily be construed as preferred or advantageous over other embodiments. The detailed description includes specific details for the purpose of providing a thorough understanding of the biochemical detection systems and methods. However, it will be apparent to those skilled in the art that the biochemical detection systems and methods may be practiced without these specific details. In some instances, well-known structures and devices are shown in block diagram form in order to avoid obscuring the concepts of the biochemical detection systems and methods.

Exemplary embodiments of the biochemical detection systems and methods teach the use of a flexible probe that can be inserted into and maintained in a target tissue of the body. When connected to external apparatus as described herein, this probe can be used to detect the appearance of molecules on the surface cells of the tissue in the immediate vicinity of the end of the probe. The rate of diffusion of a reagent through a tissue is affected by the physiological effect that needs to be detected, and the rate of diffusion can be inferred from changes in the local concentration of that reagent that are detected by fluorescent emissions of that reagent.

An exemplary embodiment allows the detection of phosphatidyl serine translocation to the external surface of the cells of a malignant tumor in the vicinity of the tip of the probe. The exemplary embodiment uses FM1-43 (N-(3-triethylammoniumpropyl)-4-(4-(dibutylamino)styryl) pyridinium dibromide, Molecular Probes), a fluorophor that increases its quantum efficiency when bound to cell membranes and that increases its binding to external cell membranes in the presence of phosphatidyl serine that has been translocated to the external surface of those membranes. This detection task can be applicable to the selection of cancer chemotherapy agents based on the early detection of apoptosis induced in the malignant cells by one or more sequential trials of putative treatments given in a controlled time series. By identifying the timing and relative magnitude of the phosphatidyl serine translocation induced by each therapeutic trial, it is possible to identify the proper treatment for a given tumor.

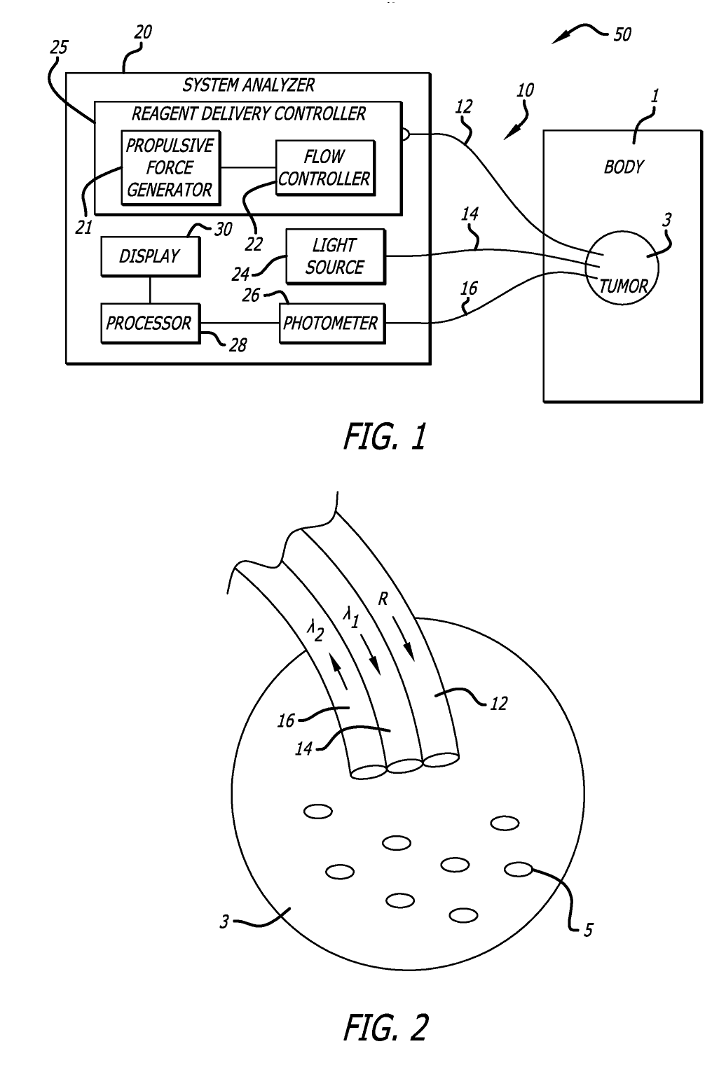

FIG. 1 illustrates an exemplary biochemical detection system 50. The system comprises a probe 10 that can be implanted into tumor 3 in the body 1 and that is connected to a system analyzer 20 that is located outside of the body 1. Probe 10 may be comprised of three separate elements bound together so as to act as a single flexible probe within the body but separately connectable to different aspects of system analyzer 20 (a detailed view of the internal (distal) end of probe 10 is shown in FIG. 2). Microcapillary 12 can be filled with reagent R and can be connected to a reagent delivery controller 25, which is comprised of a source of propulsive force 21 and flow control 22. Examples of suitable propulsive force 21 include hydrostatic pressure, which would be controllable by an electromechanical valve, or electrophoretic voltage, which would be controllable by an electronic regulator or switch. Filling the microcapillary with reagent can be accomplished by capillary action or other methods known to those skilled in the art. In some embodiments, the volume in the capillary itself may provide sufficient reagent for most applications, thus obviating the need for a reservoir.

Optical fiber 14 may be connected to light source 24, which emits wavelength λ1 that excites the fluorescence of reagent R. Light source 24 could be a monochromatic emitter such as a laser or laser diode or a polychromatic lamp equipped with a filter or monochromator, as will be obvious to anyone skilled in the art. Optical fiber 16 may be connected to photometer 26 or other light receiver known to those skilled in the art, which detects wavelength λ2 that is a fluorescent emission from reagent R when excited by λ1. Photometer 26 could be a photodiode, phototransistor or photomultiplier, as will be obvious to anyone skilled in the art. A processor 28, which can comprise a CPU chip, memory, or other devices known to those skilled in the art, can be used to determine the intensity of fluorescence over a period of time. The processor 28 may analyze other data and perform other computations that would be useful to a clinician or other user skilled in the art. The results of the fluorescent data could be displayed to the user via display 30, which may be a liquid crystal display or other display device known to those skilled in the art. In an alternative embodiment, a single optical fiber could be used for delivering light to the tissue and receiving light from the fluorescent reagent.

When reagent R is released into tumor 3, it generally diffuses away from the orifice of microcapillary 12, becoming gradually more dilute. The rate of diffusion may depend on the tendency of cells 5 comprising tumor 3 to bind reagent R to their cell membranes. When there are many binding sites and/or those binding sites have high affinity for reagent R, the concentration of R in the vicinity of the internal end of probe 10 will typically be higher than when binding is low and reagent R diffuses away more rapidly. The amount of fluorescence detected by optical fiber 16 and photometer 26 may depend on the concentration of reagent R in the immediate vicinity of the internal end of probe 10. If reagent R is chosen to be FM1-43 or anexin-V, the amount of reagent R bound to the membranes of cells 5 may be greater when phosphatidyl serine is present on their surface membranes. If the reagent R is FM1-43, the quantal efficiency of its fluorescence can be increased by its binding to the cell membranes, compared to its fluorescence when diffusing freely through the interstitial fluids. Thus, measurement of fluorescent emissions of FM1-43 at wavelength λ2 can be used to provide information about the presence of phosphatidyl serine on the surface membranes of cells 5. Phosphatidyl serine and related applications are discussed in U.S. Pat. No. 6,630,313 to Fadok et al.; U.S. Pat. No. 6,063,580 to Maiese et al.; and U.S. Pat. No. 5,939,267 to Maiese et al., each of which are hereby incorporated by reference.

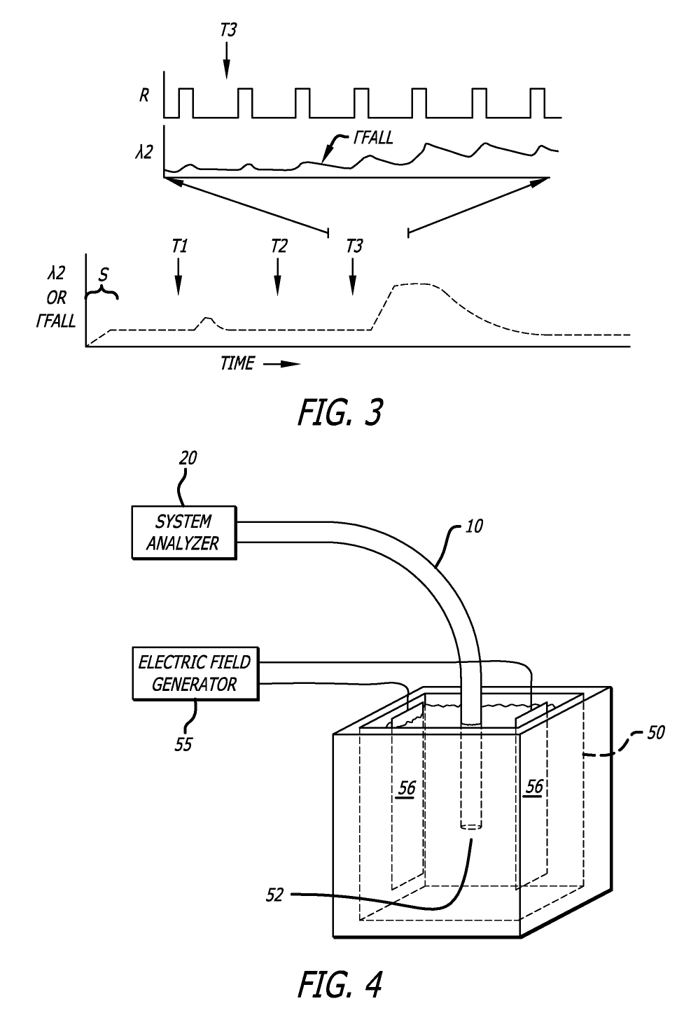

Probe 10 can be inserted some time before the commencement of the measurement time period illustrated, in order to allow it to stabilize in tumor 3. It may be advantageous to plug temporarily the distal end of microcapillary 12 with a material that prevents the diffusion of reagent R from the orifice of microcapillary 12 until measurements are to be made. Such a plug could be a gas bubble or droplet of oil or other water insoluble material that can be ejected from the orifice by propulsive force 21. In this exemplary embodiment, the flow of reagent R can be controlled in a pulsatile manner via flow control 22 as illustrated in the top trace of FIG. 3, while excitation wavelength λ1 is applied continuously via optical fiber 14 and fluorescent wavelength λ2 is measured continuously via optical fiber 16. In some, the first few aliquots of flow of reagent R may be ignored, because responses may be affected by the expulsion of a temporary plug and/or the equilibration of concentration of reagent R in the distal end of microcapillary 12 and the tissues of tumor 3 (period S in FIG. 3). Each aliquot of R will likely produce a transient rise and fall of fluorescence λ2 as illustrated in the middle trace of FIG. 3. If reagent R is bound to cells 5 of tumor 3, the transient fall rate may be much slower, which can be quantified as time constant Γfall. If the interval between successive aliquots of reagent R is shorter than Γfall, then the mean fluorescence will also increase (λ2 in FIG. 3). The bottom trace in FIG. 3 illustrates the time course of a series of experiments designed to determine the relative efficacy of three chemotherapeutic treatments T1, T2 and T3 on the cells 5 of tumor 3. Treatment T1 has a weak effect, T2 has no effect, and T3 produces a large effect. The top two traces in FIG. 3 illustrate in detail a sequence of measurements associated with the response to treatment T3, which produces a large increase in the expression of phosphatidyl serine on the surface membranes of cells 5. This reduces the diffusion rate of reagent R through tumor 3, producing a measurable increase in Γfall and an b increase in λ2.

Pulsatile delivery of reagent R may confer several advantages over continuous delivery. It may significantly reduce the total amount of reagent delivered to the tissue and reduce the possibility of accumulating a large background concentration in the tissue that could shift the dynamics and sensitivity of the assay. Measurements of the dynamics of the response such at Γfall are less likely to be affected by small movements of the probe in the tissue than measurements of instantaneous fluorescence in response to continuously infused reagent. Pulsatile delivery entails the setting of several parameters (e.g. magnitude of propulsive force, pulse duration, pulse interval) that afford opportunities to optimize the sensitivity and dynamic range of the assay for a wide range of circumstances in the tissue (e.g. density of target cells, perfusion and clearance of the reagent in the tissue, mechanical placement and tissue fixation of the probe, etc.).

Some embodiments may be adapted to detect the occurrence of many different markers on the surfaces of many different types of cells for various purposes, using various selective binding agents (e.g. antibodies, enzymes, etc.) and fluorophors. For example preferred embodiments of the present invention can be used to detect the efficiency of various therapies by analyzing markers such as cell adhesion molecules (CAMs). The following articles discuss various applications using CAMs, and are hereby incorporated by reference: Chiba and Keshishian, Neuronal Pathfinding and Recognition: Roles of Cell Adhesion Molecules, Developmental Biology 180, 424-432 (1996); Sytnyk et al., Neural cell adhesion molecule promotes accumulation of TGN organelles at sites or neuron-to-neuron contacts, Journal of Cell Biology, Vol. 159, Number 4, 649-661 (2002); Perl et al., Reduced expression of neural cell adhesion molecule induces metastatic dissemination of pancreatic β tumor cells, Nature Medicine, Vol. 5, Number 3, 286-291 (1999). Moreover, various embodiments may be employed to detect other changes in the diffusibility of a reagent through tissue such as might be caused by changes in the structure, relative volume or constituent elements of interstitial fluid. These may include, but are not limited to, changes in ionic pumps in cell membranes, osmolality of blood and interstitial fluids, adhesion between cells, and composition of basement membranes surrounding cells.

Some embodiments may utilize various methods known to those skilled in the art to excite and measure the fluorescent response, including pulsatile and sinusoidal modulation of excitation and persistence and phase-delay of fluorescence.

In an exemplary embodiment, the probe comprises only one optical fiber, which can be used both to deliver the excitation and detect the fluorescence. In such an embodiment, the system analyzer can be equipped with conventional photonic technology for beam-splitting and filtering. In alternative embodiments, the probe could be equipped with additional microcapillary channels for infusing therapeutic agents locally in the tissue around the end of the probe or electrodes for producing or detecting other physiological responses.

The probe can be implanted by injection. Moreover, in some embodiments the probe can be left in situ for many days to facilitate a series of measurements of responses to a variety of pharmacological treatments or experiments. In an exemplary embodiment, the probe can remain passive during healing from the initial insertion and can then be activated when measurements are desired. In alternative embodiments, the probe can be used during surgical procedures, including but not limited to, for example, biopsies and endoscopic procedures.

The amount and timing of release of the fluorescent reagent can be precisely controlled to facilitate detection of responses under a wide range of ambient conditions at the tip of the probe. In some embodiments, the total amount of fluorescent reagent delivered to the body can be minimized, facilitating the use of reagents that may be toxic in larger, systemic doses.

In an exemplary embodiment, the measurements can be dominated by the responses of a small, precisely located and constant volume of tissue in the immediate vicinity of the tip of the probe. Furthermore, the fluorescence of the reagent in the tissue is readily separated from the fluorescence of the reagent being delivered to the tissue and from the light used to excite the fluorescence of the reagent in the tissue.

Another embodiment can be used to optimize the mechanical design and selection of functional parameters for the invention for a particular application without requiring a living subject. It can also be used to screen therapeutic agents using living cells cultured in vitro. For example, such embodiments simulate the conditions of a three-dimensional tissue with cells trapped in relative position by an extracellular matrix that permits diffusion of small molecules. A suspension of the cells of interest can be photopolymerized into a loose, hydrophilic matrix 52 of polyethylene glycol (PEG). Before photopolymerization, the suspension plus PEG can be poured into chamber 50, illustrated in FIG. 4, with probe 10 suspended in the middle of chamber 50. In order to produce reliable and precisely timed translocation of phosphatidyl serine in the membranes of the cells, the walls of chamber 50 can be fitted with electrodes 52 that can be used to administer intense, brief electrical fields from generator 55 that are known to produce such translocations (Vernier et al., 2004, appended). The microcapillary 12 of the probe 10 can be filled with reagent R, a fluorescent material that binds selectively to phosphatidyl serine such as FM1-43 or annexin-V-fluorophor. As described previously and illustrated in FIGS. 1-3, the external end of microcapillary 12 can be attached to a propulsive means such as a hydrostatic pressure or an electrophoretic voltage whose strength and timing can be experimentally controlled, and optical fibers 14 and 16 can be used to excite and to measure fluorescence of reagent R, respectively.

In an exemplary in vitro embodiment, pulses of propulsion at a regular frequency can be applied to the microcapillary to extrude fixed aliquots of the fluorescent reagent R. The rise and fall of fluorescence in the cellular matrix at the end of the probe can be measured in response to each aliquot. When a steady-state has been reached, the electrodes can be used to apply a brief field known to cause phosphatidyl serine translocation. This is expected to produce a change in the rise and fall pattern of fluorescence in response to the aliquots of fluorescent reagent R. Because reagent R tends to bind to the phosphatidyl serine on the cell surfaces, its rate of diffusion away from the probe may be substantially slowed, producing a longer time constant for the fall of the fluorescence recorded for each aliquot (Γfall) and an increase in mean fluorescence λ2. The responses to this known method for producing controlled translocation of phosphatidyl serine uniformly in a population of cells may be compared to responses to putative chemotherapeutic agents to which the cells can be exposed. Such agents can be incorporated into matrix 52 when it is initially polymerized or introduced by local infusion into or bulk diffusion through matrix 52. Exemplary embodiments can utilize various means of controlling the timing of exposure of the cells to active agents known in the art, such as electrophoresis and photoactivation, for example.

Embodiments of the biochemical detection systems and methods can be adapted to detect and/or analyze the effectiveness of various chemotherapy agents on cancer cells and tumors. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof. Chemotherapeutic agents and methods of administration, dosages, etc. are well known to those of skill in the art (see for example, the “Physicians Desk Reference”, Goodman & Gilman's “The Pharmacological Basis of Therapeutics” and in “Remington's Pharmaceutical Sciences”, incorporated herein by reference in relevant parts).

Agents or factors suitable for analysis may include any chemical compound that induces DNA damage when applied to a cell. Chemotherapeutic agents to analyze include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing.

An exemplary embodiment can be adapted to analyze the effectiveness of alkylating agents that directly interact with genomic DNA to prevent the cancer cell from proliferating. Preferred embodiments can be used to detect the effectiveness of chemotherapeutic alkylating agents that affect all phases of the cell cycle. Alkylating agent that can be analyzed may include, but are not limited to, a nitrogen mustard, an ethylenimene, a methylmelamine, an alkyl sulfonate, a nitrosourea or a triazines. They include but are not limited to: busulfan, chlorambucil, cisplatin, cyclophosphamide (cytoxan), dacarbazine, ifosfamide, mechlorethamine (mustargen), and melphalan.

Another exemplary embodiment can be adapted to analyze the effectiveness of chemotherapeutic antimetabolites that disrupt DNA and RNA synthesis. Various categories of antimetabolites that may be analyzed include, but are not limited to, folic acid analogs, pyrimidine analogs and purine analogs and related inhibitory compounds. Specific antimetabolites that may be analyzed include but are not limited to, 5-fluorouracil (5-FU), cytarabine (Ara-C), fludarabine, gemcitabine, and methotrexate.

Other exemplary embodiments can be adapted to analyze chemotherapeutic agents originally isolated from a natural source. Such compounds, analogs and derivatives thereof may be isolated from a natural source, chemically synthesized or recombinantly produced by any technique known to those of skill in the art. Natural products to be analyzed include but are not limited to such categories as mitotic inhibitors, antitumor antibiotics, enzymes and biological response modifiers.

Further exemplary embodiments can be adapted to analyze mitotic inhibitors such as plant alkaloids and other natural agents that can inhibit either protein synthesis required for cell division or mitosis. Mitotic inhibitors that may be analyzed include but are not limited to, for example, docetaxel, etoposide (VP16), teniposide, paclitaxel, taxol, vinblastine, vincristine, and vinorelbine. Taxoids, which are a class of related compounds isolated from the bark of the ash tree, Taxus brevifolia, can also be analyzed. Taxoids include but are not limited to compounds such as docetaxel and paclitaxel. Furthermore, embodiments can be adapted to analyze the effectiveness of vinca alkaloids, including but not limited to compounds such as vinblastine (VLB) and vincristine.

Another exemplary embodiment can be adapted to analyze the effectiveness of antitumor antibiotics that interfere with DNA by chemically inhibiting enzymes and mitosis or altering cellular membranes. Examples of antitumor antibiotics that can be analyzed by such preferred embodiments include but are not limited to, bleomycin, dactinomycin, daunorubicin, doxorubicin (Adriamycin), plicamycin (mithramycin) and idarubicin.

Further exemplary embodiments can be adapted to analyze the effectiveness of hormones used to kill or slow the growth of cancer cells. For example, corticosteroid hormones, such as prednisone and dexamethasone, may be detected and/or analyzed. Furthermore, embodiments may be adapted to analyze the effectiveness of: progestins (such as hydroxyprogesterone caproate, medroxyprogesterone acetate, and megestrol acetate); estrogens (such as diethylstilbestrol and ethinyl estradio); antiestrogens (such as tamoxifen); androgens (such as testosterone propionate and fluoxymesterone); antiandrogens (such as flutamide); and gonadotropin-releasing hormone analogs (such as leuprolide).

Additional chemotherapeutic agents that may be analyzed include, but are not limited to: platinum coordination complexes, anthracenedione, substituted urea, methyl hydrazine derivative, adrenalcortical suppressant, amsacrine, L-asparaginase, and tretinoin, can also be analyzed alternative preferred embodiments. Furthermore, embodiments may also analyze the effectiveness of anti-angiogenic agents including but not limited to angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16 K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4, and minocycline.

The biochemical detection systems and methods can also be adapted to analyze various biomolecules associated with cellular metabolism and/or structure, cancer and/or effective treatment of cancer cells. Such biomolecules include but are not limited to: lipids, carbohydrates, organic or inorganic molecules, nucleic acids, proteins, metabolites, functional states of proteins, enzymes, cytokines, chemokines, and other factors, e.g. growth factors, such factors include GM-CSF, G-CSF, M-CSF, TGF, FGF, EGF, TNF-α, GH, corticotropin, melanotropin, ACTH, extracellular matrix components, surface membrane proteins, such as integrins and adhesins, soluble or immobilized recombinant or purified receptors, and antibodies against receptors or ligand mimetics.

Further biochemical detection systems and methods can analyze other parameters of interest, including detection of cytoplasmic, cell surface or secreted biomolecules, frequently biopolymers, such as polypeptides, polysaccharides, polynucleotides, and lipids. Cell surface and secreted molecules are a parameter type as these mediate cell communication and cell effector responses and can be more readily assayed. In one embodiment, parameters include specific epitopes. Epitopes are frequently identified using specific monoclonal antibodies or receptor probes. In some cases the molecular entities comprising the epitope are from two or more substances and comprise a defined structure; examples include combinatorially determined epitopes associated with heterodimeric integrins. A parameter may be detection of a specifically modified protein or oligosaccharide, e.g. a phosphorylated protein, such as a STAT transcriptional protein; or sulfated oligosaccharide, or such as the carbohydrate structure Sialyl Lewis x, a selectin ligand. The presence of the active conformation of a receptor may comprise one parameter while an inactive conformation of a receptor may comprise another. A parameter may be defined by a specific monoclonal antibody or a ligand or receptor binding determinant. Parameters may include the presence of cell surface molecules such as CD antigens (CD1-CD247), cell adhesion molecules, selectin ligands, such as CLA and Sialyl Lewis x, and extracellular matrix components. Parameters may also include the presence of secreted products such as lymphokines, including IL-2, IL-4, IL-6, growth factors, etc. (Leukocyte Typing VI, T. Kishimoto et al., eds., Garland Publishing, London, England, 1997); Chemokines in Disease: Biology and Clinical Research (Contemporary Immunology), Hebert, Ed., Humana Press, 1999. For activated T cells parameters that can be detected and/or analyzed by the biochemical detection systems and methods may include IL-1R, IL-2R, IL4R, IL-12Rβ, CD45RO, CD49E, tissue selective adhesion molecules, homing receptors, chemokine receptors, CD26, CD27, CD30 and other activation antigens. Additional parameters that are modulated during activation include MHC class II ; functional activation of integrins due to clustering and/or conformational changes; T cell proliferation and cytokine production, including chemokine production. Of particular importance is the regulation of patterns of cytokine production, the best-characterized example being the production of IL-4 by Th2 cells, and interferon-γ by Th1 T cells.

In an exemplary embodiment, the sequential timing of candidate treatments can be optimized based on variance in time delays of apoptotic responses.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the biochemical detection systems and methods. Thus, the biochemical detection systems and methods are not intended to be limited to the embodiments shown herein but is to be accorded the widest scope consistent with the principles and novel features disclosed herein.

What is claimed is:

1. A system for detecting molecules within a tissue, comprising:
a) a probe comprising:
i. at least one reagent delivery device that is configured for insertion into tissue;
ii. at least one optical fiber that is configured for insertion into tissue; and
b) a system analyzer comprising:
ii. a light source configured to deliver light of a first wavelength through the probe to the tissue;
iii. a reagent delivery controller configured for pulsatile delivery of fluorescent reagent through the probe to the tissue; and
iii. a light receiver configured to receive and analyze light of a second wavelength, different from the first wavelength, from the probe.

2. The system of claim 1, wherein the reagent delivery controller comprises a capillary tube.

3. The system of claim 1, wherein the reagent delivery controller comprises a flow controller to control the amount of reagent to be delivered.

4. The system of claim 1, wherein the reagent delivery controller comprises a controller to control the timing of the delivery of the fluorescent reagent.

5. The system of claim 1, wherein the reagent delivery controller comprises a propulsive force generator.

6. The system of claim 1, wherein the fluorescent reagent comprises annexin-V and/or FM1-43.

7. The system of claim 1, wherein the fluorescent reagent comprises at least one immunofluorescent agent capable of interacting with at least one NCAM.

8. The system of claim 1, wherein the fluorescent reagent comprises FM1-43 (N-(3-triethylammoniumpropyl)-4-(4-dibutylamino)styryl) pyridinium dibromide.

9. The system of claim 1, wherein the fluorescent reagent binds to a molecule at the surface of a cell membrane.

10. The system of claim 9, wherein the molecule at the surface of a cell membrane is indicative of apoptosis.

11. The system of claim 9, wherein the molecule is phosphatidyl serine.

12. The system of claim 1, wherein the light receiver comprises a photometer.

13. The system of claim 12, wherein the photometer comprises at least one of photodiode; phototransistor or photomultiplier.

14. The system of claim 1, wherein the tissue is a tumor.

15. The system of claim 4, wherein the timing controller can control the timing of delivery of the fluorescent reagent based upon response from a volume of a tissue in the vicinity of the probe.

16. The system of claim 1, wherein the pulsatile delivery comprises pulses of propulsion at stable frequencies.

17. The system of claim 1, further comprising a processor comprising a CPU and memory.

18. The system of claim 1, further comprising a display device.

19. A method of detecting apoptosis, comprising the steps of:
a) delivering a plurality of pulses of a controlled quantity of fluorescent reagent at a controlled frequency through a probe into a tissue;
b) delivering light of a first wavelength through the probe to the tissue;
c) receiving, through the probe, light of at least a second wavelength, different from the first wavelength, from the fluorescent reagent and/or the tissue; and
d) analyzing the received light.

20. The method of claim 19, wherein the pulses of the fluorescent reagent are delivered through a capillary tube of the probe.

21. The method of claim 19, wherein the quantity of the fluorescent reagent is controlled through a flow controller.

22. The method of claim 21, wherein the quantity of the fluorescent reagent is controlled based on response from a volume of a tissue in the vicinity of the probe.

23. The method of claim 19, wherein the timing of delivery of fluorescent reagent through the probe is controlled by a timing controller.

24. The method of claim 19, wherein the delivery of fluorescent reagent is achieved by providing a propulsive force.

25. The method of claim 19, wherein the fluorescent reagent comprises annexin-V and/or FM1-43.

26. The method of claim 19, wherein the fluorescent reagent comprises at least one immunofluorescent agent that binds with at least one NCAM.

27. The method of claim 19, wherein the fluorescent reagent comprises FM1-43 (N-(3-triethylammoniumpropyl)-4-(4-dibutylamino)styryl) pyridinium dibromide.

28. The method of claim 19, wherein the fluorescent reagent binds with a molecule at the surface of a cell membrane.

29. The method of claim 28, wherein the molecule at the surface of a cell membrane is indicative of apoptosis.

30. The method of claim 29, wherein the molecule is phosphatidyl serine.

31. The method of claim 19, wherein the received light is analyzed by a photometer.

32. The method of claim 31, wherein the photometer comprises at least one of a photodiode; phototransistor or photomultiplier.

33. The method of claim 19, wherein the tissue is a tumor.

34. The method of claim 19, wherein the time between delivered pulses of fluorescent reagent is shorter than the fall time of fluorescence of the fluorescent reagent.

35. The method of claim 19, further comprising sinusoidal modulation of excitation, persistence and/or phase-delay of fluorescence.

36. The method of claim 19, wherein the analysis of the received light further comprises processing signals through a processor comprising a CPU, and a memory.

37. The method of claim 19, further comprising displaying the result of the analysis of the received light through a display device.

38. The method of claim 19, further comprising determining the mean fluorescence of the delivered fluorescent reagent over time.
